(19) United States
(12) Patent Application Publication
(10) Pub. No.: US 2011/0160213 A1
(43) Pub. Date: Jun. 30, 2011

(54) PHARMACEUTICAL COMPOSITIONS FOR THE TREATMENT OF INFLAMMATORY AND ALLERGIC DISORDERS

(75) Inventors: Ulhas Dhuppad, Nashik (IN); Ganga Srinivas Arra, Hyderabad (IN)

(73) Assignee: Glenmark Pharmaceuticals, S.A., La Chaux-de-Fonds (CH)

(21) Appl. No.: 12/525,049

(22) PCT Filed: Jan. 31, 2008
(86) PCT No.: PCT/IB2008/000217

§ 371 Date: Jul. 29, 2009

Related U.S. Application Data
(60) Provisional application No. 60/890,693, filed on Feb. 20, 2007.

(30) Foreign Application Priority Data
Feb. 1, 2007 (IN) 190/MUM/2007

(51) Int. Cl. A61K 31/5025; A61K 31/4375; A61P 29/00; A61P 37/08

(52) U.S. Cl. 514/248; 514/293

(57) ABSTRACT

The present invention relates to the pharmaceutical compositions, more particularly to the pharmaceutical compositions comprising novel phosphodiesterase type 4 (PDE4) inhibitors, and their use in treating allergic and inflammatory disorders.

### PRIORITY DOCUMENT(S)

This patent application is the U.S. National Phase of International patent application no. PCT/IB2008/000217, filed Jan. 31, 2008, which claims priority to Indian provisional patent application no. 190/MUM/2007 filed on Feb. 1, 2007, and U.S. provisional patent application No. 60/890,693, filed on Feb. 20, 2007, all of which are hereby incorporated reference.

### BACKGROUND OF THE INVENTION

1. Technical Field

The present invention relates to a pharmaceutical composition comprising a phosphodiesterase type 4 inhibitor, and its use in treating allergic and inflammatory disorders.

2. Introduction

Phosphodiesterase type 4 (“PDE4”) inhibitors are useful in the treatment of allergic and inflammatory diseases. PDE4 inhibitors are particularly useful for the treatment of asthma and chronic obstructive pulmonary disease “COPD”).

Severe lung diseases, including asthma and COPD are characterized by airway inflammation. Events leading to airway obstruction include edema of airway walls, infiltration of inflammatory cells into the lung, production of various inflammatory mediators and increased mucous production.

WO 2006/064355, hereinafter “WO '355”, describes various PDE4 inhibitors, and their use in allergic and inflammatory disorders, including asthma and COPD. The PDE4 inhibitors described in WO '355 have the chemical structure (Formula I).

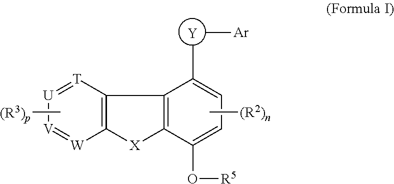

In particular, WO '355 discloses 3,5-dichloro-4-(6-difluoromethoxybenzo[4,5]furo[c]pyridin-9-ylcarboxamido)-1-pyridiniumolate, sodium salt (Example 27) as a PDE4 inhibitor.

### SUMMARY OF THE INVENTION

The inventors have discovered that the PDE4 inhibitor 3,5-dichloro-4-(6-difluoromethoxybenzo[4,5]furo[3,2-c]pyridin-9-ylcarboxamido)-1-pyridiniumolate, sodium salt exhibits poor solubility in water and that its solubility varies with (see Table 1 in the examples below). The present invention provides formulations and methods of preparing them which improve the solubility of this PDE4 inhibitor, as well as the other PDE4 inhibitors disclosed in WO '355, and minimize changes in their solubilities due to pH. As a result, the formulations of the present invention provide more rapid and uniform dissolution and enhanced bioavailability of the PDE4 inhibitor.

One embodiment of the present invention is a pharmaceutical composition comprising a PDE4 inhibitor, at least one solubility enhancing agent, and optionally one or more pharmaceutically acceptable excipients. The PDE4 inhibitor is selected from compounds having, the formula

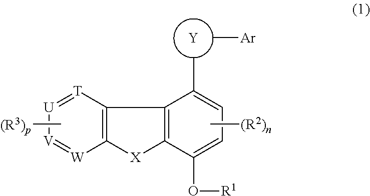

wherein
each occurrence of R¹, R² and R³ may be same or different and are independently hydrogen, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heteroarylalkyl, substituted or unsubstituted heterocyclic group, substituted or unsubstituted heterocyclylalkyl, —NR₅R⁶, —C(=L)-R⁵, —C(O)—R⁵, —C(O)O—R⁵, —C(O)NR⁵R⁶, —S(O)ₘ—R⁵, —S(O)ₘ—NR⁵R⁶, nitro, —OH, cyano, oxo, formyl, acetyl, halogen, —OR⁵, —SR₅, or a protecting group, or when two R² or two R³ substituents are ortho to each other, the two substituents may be joined to a form a 3-7 member optionally substituted saturated or unsaturated cyclic ring, which may optionally include up to two heteroatoms selected from O, NR⁵ or S;
each occurrence of R⁵ and R⁶ may be same or different and are independently hydrogen, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heteroarylalkyl, substituted or unsubstituted heterocyclic group, substituted or unsubstituted heterocyclylalkyl, nitro, halo, —OH, cyano, —C(O)O—R^{a}, —C(O)O—R^{a}, —C(O)NR^{a}R^{b}, —S(O)ₘ—R^{a}, —S(O)ₘ—NR^{a}R^{b}, —C(═NR^{a})—R^{b}, —C(═NR^{a})—NR^{a}R^{b}, —C(═S)—NR^{a}R^{b}, —C(═S)—R^{a}, —N═C(R^{a}R^{b}), —NR^{a}R^{b}, —OR^{a}, —SR^{a}, or a protecting group or R⁵ and R⁶ may be joined together with the atom to which they are attached to form a 3-7 member optionally substituted saturated or unsaturated cyclic ring, which may optionally include up to two heteroatoms selected from O, NR^{a} or S;
each occurrence of R^{a} and R^{b} may be same or different and are independently hydrogen, substituted or unsubstituted alkyl, substituted or unsubstituted alkenyl, substituted or unsubstituted alkynyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted aryl, substituted or unsubstituted arylalkyl, substituted or unsubstituted heteroaryl, substituted or unsubstituted heteroarylalkyl, substituted or unsubstituted heterocyclic group, substituted or unsubstituted heterocyclylalkyl, nitro, —OH, cyano, formyl, acetyl, halogen, a protecting group, —C(O)—R^{a}, —C(O)O—R^{a}, —C(O)NR^{a}R^{b}, —S(O)ₘ—R^{a}, —S(O)ₘ—NR^{a}R^{b}, —NR^{a}R^{b}, —OR^{a}, or —SR^{a};
Ar is substituted or unsubstituted aryl, substituted or unsubstituted arylalkyl, substituted or unsubstituted heterocyclic ring, substituted or unsubstituted heterocycloalkyl, substituted or unsubstituted heteroaryl ring, or substituted or unsubstituted heteroarylalkyl;
L is O, S or NR^{a}, where R^{a} is as defined above;
n is an integer from 0 to 2;
p is an integer from 0 to 4;
T, U, V and W are each independently C, C═O, N, NR^{a}, O or S, with the proviso that at least one of T, U, V and W are N, NR^{a}, O or S, where R^{a} is as defined above;
each dotted line [----] in the ring represents an optional bond;
X is O, S(O)ₘ or NR^{b}, where R^{b} is as defined above;
each occurrence of in is independently 0, 1 or 2;
Y is —C(O)NR⁴—, —NR⁴SO₂, —SO₂NR⁴— or —NR⁴C(O)—;
R⁴ is hydrogen, substituted or unsubstituted alkyl, hydroxyl, —OR^{a} (wherein R^{a} is defined above), substituted or unsubstituted cycloalkyl, substituted or unsubstituted cycloalkylalkyl, substituted or unsubstituted aryl, substituted or unsubstituted arylalkyl, substituted or unsubstituted heterocyclic ring, substituted or unsubstituted heterocycloalkyl, substituted or unsubstituted heteroaryl ring or substituted or unsubstituted heteroarylalkyl, or a N-oxide thereof or a pharmaceutically acceptable salt thereof.

Preferred PDE4 inhibitors include, but are not, limited to, N9-(3,5-dicholoro-4 pyridyl)-6-difluoromethoxybenzo[4,5]furo[2,3-d]pyridazine-9-carboxamide, 3,5-dichloro-4-(6-difluoromethoxybenzo[4,5]furo[3,2-c]pyridin-9-ylcarboxamido)-1-pyridiniumolate, and pharmaceutically acceptable salts thereof. The PDE4 inhibitor can be in crystalline or amorphous form, or some of the PDE4 inhibitor can be in crystalline form with the remainder in amorphous form.

Suitable solubility enhancing agents include, but are not limited to, surfactants, clathrates, buffers that control the microenvironment pH of the active agent, and agents that enables the formation of a solid dispersion of the active agent.

According to a preferred embodiment, the pharmaceutical composition comprises a therapeutically effective amount of the PDE4 inhibitor. These pharmaceutical compositions are useful for treating allergic and inflammatory disorders. The pharmaceutical composition can be in the form of a tablet, capsule, granules, beads, pellets, powder, or dry syrup for suspension.

In one embodiment, the pharmaceutical composition releases more than about 30%, about 70 or about 90% of the PDE4 inhibitor in an aqueous media. More particularly, such rapid release of the PDE4 inhibitor has been observed in 0.1 N HCl (900 ml) when tested for in vitro dissolution using a USP type II apparatus at a rotation speed of about 50 rpm and a temperature of about 37° C. in about 30 minutes from the start of the test.

In a further aspect, the present invention provides a process for preparing, a pharmaceutical composition comprising the aforementioned PDE4 inhibitor. These processes can include dry or wet granulation, direct compression, powder mixing, pellet formation, or liquid mixing.

### DETAILED DESCRIPTION OF THE INVENTION

### Definitions

For the purpose of present invention, PDE4 inhibitors, may be hereafter represented by the term “active agent” or “drug”. Preferred PDE4 inhibitors for use in the present invention include, but are not limited to, N9-(3,5-dicholoro-4 pyridyl)-6-difluoromethoxybenzo[4,5]furo[2,3-d]pyridazine-9-carboxamide, 3,5-dichloro-4-(6-difluoromethoxybenzo[4,5]furo[3,2-c]pyridin-9-ylcarboxamido)-1-pyridiniumolate and pharmaceutically acceptable salts thereof, are referred to hereinafter by the term “the agent”. For ease of understanding, the compound 3,5-dichloro-4-(6-difluoromethoxybenzo[4,5]furo[3,2-c]pyridin-9-ylcarboxamido)-1-pyridiniumolate or a pharmaceutically acceptable salt thereof is referred to as “the agent I”. While the compound N 9-(3,5-dicholoro-4-pyridyl)-6-difluoromethoxybenzo[4,5]furo[2,3-d]pyridazine-9-carboxamide or a pharmaceutically acceptable salt thereof is referred to as “the agent 2”.

The term “treating” or “treatment” of a state, disorder or condition as used herein means: (1) preventing or delaying the appearance of clinical symptoms of the state, disorder or condition developing in a mammal that may be afflicted with or predisposed to the state, disorder or condition but does not yet experience or display clinical or sub clinical symptoms of the state, disorder or condition, (2) inhibiting the state, disorder or condition, i.e., arresting or reducing the development of the disease or at least one clinical or sub clinical symptom thereof, or (3) relieving the disease, i.e., causing regression of the state, disorder or condition or at least one of its clinical or sub clinical symptoms. The benefit to a subject to be treated is either statistically significant or at least perceptible to the subject or to the physician

The term “effective amount” as used herein means the amount of a compound that, when administered to a mammal for treating a state, disorder, condition or causing an action, e.g., treatment of inflammation, sufficient to effect such treatment or action. The “effective amount” will vary depending on the compound, the disease and its severity and the age, weight, physical condition and responsiveness of the mammal to be treated.

By “pharmaceutically acceptable” is meant those salts and esters which are, within the scope of sound medical judgment, suitable for use in contact with the tissues of humans and lower animals without undue toxicity, irritation, and allergic response, commensurate with a reasonable benefit/risk ratio, and effective for their intended use. Representative acid additions salts include the hydrochloride, hydrobromide, sulphate, bisulphate, acetate, oxalate, valerate, oleate, palmitate, stearate, laurate, borate, benzoate, lactate, phosphate, tosylate, mesylate, citrate, maleate, fumarate, succinate, tartrate, ascorbate, glucoheptonate, lactobionate, and lauryl sulphate salts. Representative alkali or alkaline earth metal salts include the sodium, calcium, potassium and magnesium salts.

The term “subject” as used herein refers to mammalian animals, including humans.

The pharmaceutical composition can be a dosage form to facilitate administration of the PDE4 inhibitor to a subject. Suitable oral dosage forms includes tablets, capsules, powders, granules, pellets/beads, liquid filled capsules, dry syrups for suspension, and liquid formulations such as syrups, suspensions, semisolids, and gels. Such compositions may be prepared by various techniques such as dry or wet granulation, direct compression, powder mixing, pellet formation, and liquid mixing as known to a person skilled in the formulation art.

The term “solubility enhancing agent” refers to an agent that increases the solubility or dissolution of the PDE4 inhibitor when compounded together to form a composition. Such solubility enhancing agents include, but are not limited to, surfactants, clathrates, buffering agents that control the microenvironment pH of the PDE4 inhibitor, and agents that enable formation of a solid dispersion of the active agent, and any combination of any of the foregoing. Exemplary solubility enhancing agents include, without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., TWEEN®), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxyl propylcellulose, hydroxypropylmethylcellulose phthalate, noncrystalline cellulose, magnesium aluminum silicate, triethanolamine, polyvinyl alcohol, and polyvinylpyrrolidone (PVP), hydroxypropyl cellulose, hydroxypropylmethyl cellulose, clathrates with cyclodextrins like beta cyclodextrin and their derivatives, CREMOPHOR®, TYLOXAPOL®, GELUCIRE®, sodium laurel sulphate, buffers like citrates, carbonates and bicarbonates, acetates and the like, and combinations thereof and other such materials known to those of ordinary skill in the art.

These pharmaceutical compositions exhibit improved solubility and dissolution of the PDE4 inhibitor, which results in enhanced bioavailability. Various approaches to formulate such compositions are described in the context of the present invention as well as the references cited herein.

According to one embodiment, the pharmaceutical composition is a dry powder for suspension which comprises in addition to the PDE4 inhibitor and solubility enhancing agent, at least one excipient selected from binders, diluents, suspending agents, dispersing agents, sweetener, and coloring agents.

Suitable suspending agents include swellable materials, such as gums and hydroxypropyl methylcellulose (“HPMC”). When HPMC contacts water, it forms a complex polymeric structure which enhances the viscosity of the resulting solution. The degree of polymerization depends upon the viscosity grade of the HPMC. Due to the enhanced viscosity of the solution, the PDE4 inhibitor is more uniformly distributed in the medium and remains suspended in the liquid medium without significant sedimentation.

The pharmaceutical composition of the present invention containing the PDE4 inhibitor and a solubility enhancing agent can be formulated with one or more pharmaceutically acceptable excipients. The amount of the additional pharmaceutically acceptable excipients generally varies from about 10% to about 90% by weight, based on the total weight of the composition.

According, to another embodiment, the pharmaceutical composition is in the form of a tablet or capsule. The pharmaceutical composition comprises in addition to the PDE4 inhibitor and solubility enhancing agent, at least one excipient selected from fillers, binders, disintegrants, and lubricants. The tablet may be an orally disintegrating or fast dissolving tablet.

Suitable fillers include, fix example, lactose, sugar, starches, modified starches, mannitol, sorbitol, inorganic salts, cellulose, cellulose derivatives (e.g., microcrystalline cellulose), calcium sulfate, xylitol and lactitol.

Suitable binders include, for example, polyvinyl pyrrolidone, lactose, starches, modified starches, sugars, gum acacia, gum tragacanth, guar gum, pectin, wax hinders, microcrystalline cellulose, methylcellulose, carboxymethyl cellulose, hydroxypropyl methylcellulose, hydroxyethyl cellulose, hydroxypropyl cellulose, copolyvidone, gelatin and sodium alginate.

Suitable disintegrants include, for example, crosscarmellose sodium, crospovidone, polyvinyl pyrrolidone, sodium starch glycolate, corn starch, microcrystalline cellulose, and hydroxypropyl methyl cellulose and hydroxy propyl cellulose.

Suitable lubricants include, for example, magnesium stearate, stearic acid, palmitic acid, calcium stearate, talc, carnauba wax, hydrogenated vegetable oils, mineral oil, polyethylene glycols and sodium stearyl fumarate.

Additional excipients which may be incorporated into the pharmaceutical composition include, for example, preservatives, stabilizers, anti-oxidants, silica flow conditioners, antiadherents and glidants.

Other suitable fillers, binders, disintegrants, lubricants and additional excipients which may be used are described in Handbook of Pharmaceutical Excipients, 2^{nd} Edition, American Pharmaceutical Association; The Theory and Practice of Industrial Pharmacy, 2^{nd} Edition, Lachman, Leon, 1976; Pharmaceutical Dosage Forms: Tablets Volume 1, 2^{nd} Edition, Lieberman, Hebert A., et al, 1989; Modern Pharmaceutics, Banker, Gilbert and Rhodes, Christopher T, 1979; and Remington's Pharmaceutical Sciences, 15^{th} Edition, 1975, all of which are hereby incorporated by reference.

In one embodiment, the pharmaceutical composition releases more than about 50%, about 70%, or about 90% of the PDE4 inhibitor within 30 minutes of placement of the pharmaceutical composition in an aqueous solution containing 0.1 N HCl (900 ml) at 37° C. in a USP type II apparatus at a rotation speed of about 50 rpm.

The pharmaceutical composition can have a moisture content less that about 6% w/w, or about 4% w/w as determined using a Karl-Fisher moisture analyzer (Karl-Fisher titrator; Model 794; METROHM®, Switzerland).

In a further aspect, the present invention provides a process for preparing a pharmaceutical composition, of the present invention.

The pharmaceutical compositions of the present invention can be prepared using dry or wet granulation, direct compression, powder mixing, pellet formation, and liquid mixing. Various unit operations are involved in the process for preparing pharmaceutical compositions like weighing, dispensing, sifting, mixing or bleeding, lubricating and the like. During these unit operations, the PDE4 inhibitor and other excipients are usually handled separately or combinedly.

In one preferred embodiment, a solid dispersion containing, the PDE4 inhibitor is prepared. A solid dispersion can be prepared by dispersing a PDE4 inhibitor and a polymeric material in a suitable solvent, and evaporating the solvent. The PDE4 inhibitor is dispersed in the matrix of the polymeric material.

Alternatively, a clathrate can be brined using a cyclodextrin or an inclusion complex of the PDE4 inhibitor.

The pharmaceutical composition can also be prepared by combining a surfactant with the PDE4 inhibitor and forming a dosage form.

The pharmaceutical compositions of the present invention can be used to treat allergic and inflammatory disorders such as asthma and COPD in mammals and in particular humans.

The following examples are provided to enable one skilled in the art to practice the invention and are merely illustrative of the invention. The examples should not be read as limiting the scope of the claims.

### EXAMPLES

TABLE 1
Solubility data for 3,5-dichloro-4-(6-difluoromethoxybenzo[4,5]
furo [3,2-c]pyridin-9-ylcarboxamido)-1-pyridiniumolate,
sodium salt at about 37° C. in various media after
24 hours determined by shake flask method.

| Medium | Solubility (mg/250 ml) |
|---|---|
| Water | 0.68 |
| 0.1N HCl | 9.17 |
| Citrate buffer pH 3.0 | 6.02 |
| Acetate buffer pH 4.5 | 1.69 |
| 0.1N HCl with 1% sodium | 8.81 |
| lauryl sulphate | |
| Citrate buffer pH 3.0 with | 8.6 |
| 1% Triton X | |
| Acetate buffer pH 4.5 with | 4.81 |
| 0.1% SLS | |

### Example 1

### Dry Powder for Suspension

The agent 1 (sodium salt, 0.1 to 10%) is mixed with surfactant and polyvinyl pyrrolidone (together 1 to 10%). Subsequently mannitol (about 70-99%) and xanthum gum (0.1 to 5%) are added to the above dry mixture. Sufficient quantities of colorant and sweetner are added. Colloidal silicon dioxide up to 0-2% is added as dispersing agent. The dry powder thus produced can be mixed with purified water, before use.

### Example 2

### Tablets Comprising the Agent

The agent 1 (sodium salt) is added to ethanol:water 50:50, along with sodium lauryl sulfate and Povidone K-30. The mixture thus obtained is sprayed on a dry powder blend comprising lactose monohydrate and microcrystalline (up to 35 to 90% of total weight of composition) in a fluidized bed processor (FBP). The granules thus obtained are further dried in a FBP. The dried granules are sized and blended with crospovidone and Aerosil 200. The granules are further blended with sodium stearyl fumarate. The lubricated blend is then compressed into tablets.

### Example 3

### Formulation of Capsules

The agent 1 (sodium salt) along with Cremophor RH 40 and Povidone K-30 (both about 1 to 15%) in ethanolic solution is sprayed on pregelatinized starch (about 35-90%) in a fluidized bed processor (FBP) (Glatt). The wet mass is further dried in the GLATT FBP and sized granules are then mixed with L-HPC, and lubricated with Aerosil and sodium stearyl fumarate. The final lubricated blend is then filled in capsules.

### Example 4

### Preparation of Orally Disintegrating Tablets (ODT)

The agent 1 (sodium salt) is mixed with mannitol (Pearlitol SD 200), microcrystalline cellulose, aspartame, sodium bicarbonate, hypromellose, crospovidone, and colloidal silica. The mentioned excipients may form about 50 to 99% weight of the final tablet. Finally to the dry blend is mixed sodium stearyl fumarate for 15 minutes. The dry powder blend is then compressed into tablets. Alternatively pH modifiers in the higher range viz. magnesium silicates, dicalcium phosphate, and tribasic calcium phosphate, can be included.

### Example 5

### Composition Based on Solid Dispersion Technique

The agent 1 (sodium salt) (0.25 g) was dissolved in 400 g ethanol. To that were added 0.5 g polyethylene glycol and 1.25 g povidone K-30. This dispersion was spray dried to obtain a solid dispersion powder composition.

| Ingredients | mg per tablet |
|---|---|
| Solid dispersion of the agent 1 (as | 8.7 |
| above) | |
| Sodium starch glycollate | 15 |
| Dibasic calcium, phosphate | 150 |
| Macrocrystalline cellulose | 120.3 |
| Colloidal silicon dioxide (Aerosil 200) | 3 |
| Magnesium stearate | 3 |

The solid dispersion along with sodium starch glycolate, dibasic calcium phosphate, microcrystalline cellulose, and Aerosil 200 were blended for 5 minutes. The above mentioned blend was lubricated with magnesium stearate for 3 minutes and finally compressed into tablets.

### Example 6

### Composition Based on Complexation

The agent 1 (sodium salt) with cyclodextrin is added into ethanolic solution (50% ethanol), the solution is continuously stirred, and then adsorbed onto a lactose and microcrystalline cellulose mixture. These base granules are then mixed with HPC-L along with sodium starch glycolate and Aerosil 200 and are blended for 10 minutes. The above mentioned blend is lubricated with magnesium stearate for 3 minutes and finally compressed into tablets or filled into capsules.

### Examples 7-8

### Compositions Prepared by Granulation Process

| | Qty/tablet or capsule (mg) | |
|---|---|---|
| | Example 7 | Example 8 |
| Ingredients | (Tablet) | (Capsule) |
| The agent 1 (sodium salt) | 1.1 | 1.1 |
| Ethanol# | 75 | 150 |
| Water# | 75 | — |
| Polyvinyl pyrrolidone K 30 (Plasdone K 30) | 5 | 5 |
| Sodium lauryl sulphate (SLS) | 5 | — |
| Cremophor RH 40 | — | 5 |
| Lactose monohydrate | 120.9 | — |
| Microcrystalline cellulose (MCC) | 150 | — |
| Crospovidone | 12 | — |
| Pregelatinized starch | — | 270 |
| Hydroxypropyl cellulose (HPC) | — | 12 |
| Colloidal silicon dioxide | 3 | 3 |
| Sodium stearyl fumarate | 3 | 3 |
| Tablet or Capsule weight | 300 | 300 |

#Evaporates during processing.

### Manufacturing Process:

1. The agent was added to ethanol (and water in case of Example 7), followed by Plasdone and SLS (in case of Example 7) and Cremophor (in case of Example 8) under stirring so as to form a dispersion.
2. A blend containing lactose, MCC (in case of Example 7), pregelatinized starch, and HPC (in case if Example 8) was granulated with the dispersion of step 1 using a fluid bed processor (FBP).
3. The granules were dried and blended with the remaining excipients and lubricants.
4. The granules of step 3 were compressed to form tablets using a 10 mm punch sets fitted on a compression machine in case of Example 7.
5. The granules of step 3 were filled into size “0” capsules in case of Example 8.

in vitro dissolution of compositions prepared above was evaluated in 0.1 N HCl (900 ml) using USP type II apparatus at 50 rpm and temperature of about 37° C.

Cumulative drug released (%)

| Time | Example 7 | Example 8 |
|---|---|---|
| (minutes) | (Tablet) | (Capsule) |
| 0 | 0 | 0 |
| 5 | 89 | 11 |
| 15 | 95 | 71 |
| 30 | 97 | 87 |
| 60 | 100 | 90 |

### Examples 9-10

### Compositions Prepared by Direct Compression

| | Qty/tablet (mg) | |
|---|---|---|
| Ingredients | Example 9 | Example 10 |
| The agent 1 (sodium salt) | 1.1 | 1.1 |
| Mannitol (Pearlitol SD 200) | 119.9 | 119.9 |
| Macrocrystalline cellulose (Avicel PH 102) | 150 | 150 |
| Sodium bicarbonate | 5 | 5 |
| Hydroxypropylmethyl cellulose (HPMC) | 6 | 6 |
| Sodium lauryl sulphate (SLS) | — | 5 |
| Crospovidone | 12 | 12 |
| Colloidal silicon dioxide (Aerosil 200) | 3 | 3 |
| Sodium stearyl fumarate | 3 | 3 |
| Tablet weight | 300 | 305 |

### Manufacturing Process:

1. The agent was blended with mannitol, Avicel, sodium bicarbonate, HPMC, SLS if any, and crospovidone.
2. The blend of step 1 was lubricated with Aerosil and sodium stearyl fumarate, and further compressed to form tablets using a 10 mm punch sets fitted on a compression machine.

In vitro dissolution of tablets prepared above was evaluated in 0.1 N HCl (900 ml using USP type II apparatus at 50 rpm and temperature of about 37° C.

| | |
|---|---|
| Time | Cumulative drug released (%) |

| (minutes) | Example 9 | Example 10 |
|---|---|---|
| 0 | 0 | 0 |
| 5 | 76 | 78 |
| 15 | 80 | 84 |
| 30 | 81 | 87 |
| 60 | 81 | 88 |

### Example 11

### Dry Powder Suspension Composition

| Ingredients | Qty/batch (gm) |
|---|---|
| The agent 1 (sodium salt) | 4.8 |
| Polyvinyl pyrrolidone K 30 (Plasdone K 30) | 22.5 |
| Sodium lauryl sulphate (SLS) | 22.5 |
| Mannitol 25 | 711.8 |
| Mannitol (Pearlitol SD 200) | 690 |
| Xanthan gum | 10.5 |
| Saccharin sodium | 2.3 |
| Sodium benzoate | 18 |
| Carmoisine color | 0.4 |
| Strawberry flavor | 2.3 |
| Colloidal silicon dioxide (Aerosil 200) | 15 |

### Manufacturing Process:

1. The aunt, Plasdone and SLS were sifted through ASTM # 80 sieve,
2. Xanthan gum, saccharin sodium, color and flavor were sifted through ASTM # 80 sieve.
3, Remaining excipients were sifted through ASTM # 60 sieve.
4. The materials of step 1, 2 and 3 were blinded in a blender for 15 min to obtain a powder mixture.

The moisture content of the dry powder suspension composition of Example 11 was 2.02% w/w as determined by Karl-Fisher titrator.

in vitro dissolution of the dry powder suspension composition was evaluated in (L (900 ml) using UST type II apparatus at 50 rpm and temperature of about 37° C.

| Time (minutes) | Cumulative drug released (%) |
|---|---|
| 0 | 0 |
| 5 | 99 |
| 15 | 102 |
| 30 | 103 |
| 60 | 104 |

### Example 12

### Dry Powder Suspension Composition Containing the Agent 2

| Ingredients | Qty/batch (gm) |
|---|---|
| The agent 2 (sodium salt) | 0.18 |
| Polyvinyl pyrrolidone K 30 (Plasdone K 30) | 0.88 |
| Sodium lauryl sulphate (SLS) | 0.88 |
| Mannitol 25 | 11.36 |
| Mannitol (Pearlitol SD 200) | 43.16 |
| Xanthan gum | 0.41 |
| Saccharin sodium | 0.09 |
| Sodium benzoate | 0.7 |
| Carmoisine color | 0.01 |
| Strawberry flavor | 0.09 |
| Colloidal silicon dioxide (Aerosil 200) | 0.58 |

The manufacturing process was similar to that described in Example 11.

### Example 13

### Compositions Comprising Solid Dispersion of the Agent 1 (Sodium Salt)

The agent (2.2 g) was dissolved in 300 g ethanol. To that were added 4 g polyethylene glycol and 10 g polyvinyl pyrrolidone K 30 (Plasdone K30). This dispersion was spray dried to obtain a solid dispersion powder composition.

The solid dispersion obtained was further formulated into tablets (batch size 500 tablets) as follows.

Qty/batch (g)

| Ingredients | Example 13 A | Example 13 B |
|---|---|---|
| Solid dispersion of the agent 1 | 8.8 | 8.8 |
| (as above) | | |
| Sodium starch glycollate | 15 | 15 |
| Dibasic calcium phosphate | 150 | 150 |
| Microcrystalline cellulose | 120.2 | 120.2 |
| Sodium lauryl sulphate | — | 5 |
| Colloidal silicon dioxide | 3 | 3 |
| Magnesium stearate | 3 | 3 |

The manufacturing process for tablets was similar to that described in Examples 9-10.

in vitro dissolution of tablets prepared above was evaluated in 0.1 N HCl (900 ml) using USP type II apparatus at 50 rpm and temperature of about 37° C.

| | |
|---|---|
| Time | Cumulative drug released (%) |

| (minutes) | Example 13 A | Example 13 B |
|---|---|---|
| 0 | 0 | 0 |
| 5 | 102 | 96 |
| 15 | 111 | 105 |
| 30 | 113 | 109 |
| 60 | 114 | 111 |

1. A pharmaceutical composition comprising a therapeutically effective amount of an agent selected from N9-(3,5-dicholoro-4 pyridyl)-6-difluoromethoxybenzo[4,5]furo[2,3-d]pyridazine-9-carboxamide, 3,5-dichloro-4-(6-difluoromethoxybenzo[4,5]furo[3,2-c]pyridin-9-ylcarboxamido)-1-pyridiniumolate, and pharmaceutically acceptable salts thereof, at least one solubility enhancing agent, and optionally a pharmaceutically acceptable excipient.

2. The pharmaceutical composition according to claim 1, wherein the agent is N9-(3,5-dicholoro-4 pyridyl)-6-difluoromethoxybenzo[4,5]furo[2,3-d]pyridazine-9-carboxamide or a pharmaceutically acceptable salt thereof.

3. The pharmaceutical composition according to claim 1, wherein the agent is 3,5-dichloro-4-(6-difluoromethoxybenzo[4,5]furo[3,2-c]pyridin-9-ylcarboxamido)-1-pyridiniumolate or a pharmaceutically acceptable salt thereof.

4. The pharmaceutical composition according to claim 1, wherein the solubility enhancing agent is selected from a surfactant, clathrate, buffering agent that controls the microenvironment pH of the active agent, an agent that enables formation of a solid dispersion of the active agent, and any combination of any of the foregoing.

5. The pharmaceutical composition according to claim 4, wherein the composition comprises a solid dispersion that contains 3. 5-dichloro-4-(6-difluoromethoxybenzo[4,5]furo[3,2-c]pyridin-9-ylcarboxamido)-1-pyridiniumolate or a pharmaceutically acceptable salt thereof and a polymer.

6. The pharmaceutical composition according to claim 1, wherein the composition is in the form of a tablet, capsule, granules, beads, pellets, powder, or city syrup for suspension.

7. The pharmaceutical composition according to claim 1, wherein the composition releases more than about 50 of the agent in 0.1 N HCl when tested for in vitro dissolution using a LISP type II apparatus at a rotation speed of about 50 rpm and a temperature of about 37° C. in about 30 minutes from the start of the test.

8. The pharmaceutical composition according to claim 1, wherein the composition releases more than about 70% of the agent in 0.1 N HCl when tested for in vitro dissolution using a USP type II apparatus at a rotation speed of about 50 rpm and a temperature of about 37° C. in about 30 minutes from the start of the test.

9. The pharmaceutical composition according to claim 1, wherein the composition has a moisture content less than about 6% w/w.

10. The pharmaceutical composition according to claim 9, wherein the composition has a moisture content less than about 4% w/w.
